(19) United States
(12) Patent Application Publication
(10) Pub. No.: US 2021/0338904 A1
(43) Pub. Date: Nov. 4, 2021

(54) FIBROUS MEMBRANE MATERIAL FOR SOFT TISSUE REPAIR, METHOD FOR PREPARING THE SAME, AND APPLICATION THEREOF

(71) Applicant: Shenzhen Guangyuan Biomaterial Co., Ltd., Shenzhen (CN)

(72) Inventors: Hao CHEN, Shenzhen (CN); Jia'en WU, Shenzhen (CN); Shanshan XU, Shenzhen (CN); Zhichao HAN, Shenzhen (CN)

(21) Appl. No.: 17/377,426

(22) Filed: Jul. 16, 2021

Related U.S. Application Data
(63) Continuation-in-part of application No. PCT/CN2019/129178, filed on Dec. 27, 2019.

(30) Foreign Application Priority Data
Dec. 16, 2019 (CN) 201911295633.9

(51) Int. Cl. A61L 27/58; A61L 27/56; A61L 27/22; A61L 27/54; A61L 27/18; A61L 27/36; D01D 5/00

(52) U.S. Cl. CPC A61L 27/58; A61L 27/56; A61L 27/222; A61L 27/54; A61L 27/18; A61L 2300/416; D01D 5/0015; A61L 2430/34; A61L 2300/414; A61L 2300/41; A61L 2300/406; A61L 27/3683

(57) ABSTRACT

A fibrous membrane material includes a biodegradable polymer fiber and an active material dispersed in the biodegradable polymer fiber.

### CROSS-REFERENCE TO RELATED APPLICATIONS

This application is a continuation-in-part of International Patent Application No. PCT/CN2019/129178 with an international filing date of Dec. 27, 2019, designating the United States, now pending, and further claims foreign priority benefits to Chinese Patent Application No. 201911295633.9 filed Dec. 16, 2019. The contents of all of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, Mass. 02142.

### BACKGROUND

The disclosure relates to the field of biomedical materials, and more particularly, to a fibrous membrane material for soft tissue repair, a method for preparing the same, and application thereof.

Traditional methods for treating soft tissue damages or defects include autologous transplantation and allogeneic transplantation. However, these methods are limited by the unavailability of adequate organs and complications after transplantation, including immune rejection.

In recent years, fibrous membranes prepared by electrostatic spinning technology have been widely used in nerve repair, tissue enhancement, and anti-adhesion and anti-infection for wounds. However, the fibrous membranes prepared by electrostatic spinning cannot support the tissue during the repair process and has no cell regulation ability.

### SUMMARY

The disclosure provides a fibrous membrane material for soft tissue repair. The fibrous membrane material comprises a biodegradable polymer fiber and an active material dispersed in the biodegradable polymer fiber.

The biodegradable polymer fiber of the fibrous membrane material is formed by one or more biodegradable polymers. The active material is dispersed in the biodegradable polymer fiber. The types and proportions of different biodegradable polymers in the fibrous membrane material are adjustable. The proportion of the active material with respect to the biodegradable polymer fiber is adjustable. The diameter and porosity of the biodegradable polymer fiber are adjustable. Therefore, the mechanical strength of the fibrous membrane material for soft tissue repair is adjustable, and fibroblasts are easily attached to the fibrous membrane material to proliferate, so that soft tissue damage is repaired.

In a class of this embodiment, the biodegradable polymer fiber has a diameter of 0.1-3 μm, such as 0.1 μm, 0.2 μm, 0.3 μm, 0.4 μm, 0.5 μm, 0.6 μm, 0.7 μm, 0.73 μm, 0.75 μm, 0.85 μm, 1 μm, 2 μm or 3 μm and so on.

The diameter of the biodegradable polymer fiber is controlled within a range of 0.1-3 μm. The porosity of the biodegradable polymer fiber decreases as the diameter further increases, thus reducing the ability of the tissue cells to attach to the fiber, so that the cells tend to separate and respectively aggregate again in different clusters. This increases the fiber strength and foreign bodies may be implanted, leading to severe inflammatory reactions and the failure of the tissue repair. Vice versa, as the diameter of the biodegradable polymer fiber is further reduced, the porosity of the biodegradable polymer fiber becomes too large, and the tissue cells cannot be attached to the fiber and cannot grow normally, thus reducing the fiber strength and the mechanical strength of the repaired membrane. Therefore, the repaired membrane is easy to rupture and deform.

The diameter of the biodegradable polymer fiber of the disclosure is determined by the number of electrostatic spinning nozzles, nozzle diameter, spinning voltage, spinning distance, spinning temperature, the advancing rate of a spinning solution, the shape of a spinning receiving device, the rotation speed of the spinning receiving device, a subsequent processing temperature, vacuum, and time. Owing to the reasonable adjustment of these factors, the fiber diameter is controlled to a required value.

In a class of this embodiment, the biodegradable polymer fiber has a porosity of 65-90%, such as 65%, 70%, 75%, 80%, 85% or 90%.

In a class of this embodiment, the biodegradable polymer fiber comprises a biodegradable polymer selected from the group consisting of polylactic acid, poly(lactic-co-glycolic acid) copolymer, polyethylene glycol, poly(p-dioxanone), polycaprolactone, poly(L-lactide-co-caprolactone), a triblock copolymer PLA-b-PEG-b-PLA, and a combination thereof, for example, a combination of polylactic acid and poly(lactic-co-glycolic acid) copolymer, a combination of polyethylene glycol and poly(p-dioxanone), or other arbitrary combinations.

In a class of this embodiment, the biodegradable polymer is poly(lactic-co-glycolic acid) copolymer.

In a class of this embodiment, the biodegradable polymer is a combination of the poly(lactic-co-glycolic acid) copolymer and polycaprolactone.

In a class of this embodiment, the biodegradable polymer is a combination of the poly(lactic-co-glycolic acid) copolymer and poly(p-dioxanone).

The biodegradable polymer of the fibrous membrane material is poly(lactic-co-glycolic acid), a combination of poly(lactic-co-glycolic acid) and polycaprolactone, or a combination of poly(lactic-co-glycolic acid) and poly(p-dioxanone). The fibrous membrane material is conducive to promoting the diffusion and growth of the fibroblasts, which is also conducive to the release of the active material dispersed in the fibrous membrane material.

In a class of this embodiment, the number average molecular weight of polylactic acid is 8000-70000 Da, such as 8000 Da, 10000 Da, 20000 Da, 30000 Da, 50000 Da, 60000 Da or 70,000 Da.

In a class of this embodiment, the number average molecular weight of the poly(lactic-co-glycolic acid) copolymer is 40000-100000 Da, such as 40,000 Da, 50,000 Da, 60,000 Da, 70,000 Da, 80,000 Da, 90000 Da, or 100,000 Da.

In a class of this embodiment, the number average molecular weight of polyethylene glycol is 1000-20000 Da, such as 1,000 Da, 2,000 Da, 4,000 Da, 5,000 Da, 8,000 Da, 10,000 Da, 15,000 Da, or 20,000 Da, etc.

In a class of this embodiment, the number average molecular weight of poly(p-dioxanone) is 60000-250000 Da, such as 60,000 Da, 80,000 Da, 100,000 Da, 120,000 Da, 150,000 Da, 180,000 Da, 200,000 Da, or 250000 Da.

In a class of this embodiment, the number average molecular weight of the polycaprolactone is 6000-100,000 Da, such as 60,000 Da, 70,000 Da, 80,000 Da, 90,000 Da, or 100,000 Da.

In a class of this embodiment, the molar ratio of lactide units to caprolactone units of the poly(L-lactide-co-caprolactone) is between 1: 99 and 50: 50 (for example, 1: 99, 10: 90, 30: 70, 40: 60 or 50: 50), and an average molecular weight thereof is 35000-85000 Da, such as 35000 Da, 45000 Da, 55000 Da, 65000 Da, 75000 Da or 85000 Da.

In a class of this embodiment, an average molecular weight of the triblock copolymer PLA-b-PEG-b-PLA is 60000-100000 Da, such as 60000 Da, 70,000 Da, 80,000 Da, 90000 Da, or 100,000 Da.

The number average molecular weight of each of the aforesaid polymers is controlled within a specific range. If the number average molecular weight exceeds the range, the molecular weight of the biodegradable polymer will be too high, and the implant has too polymers, leading to the rejection reaction and causing inflammation. Meanwhile, with too high molecular weight and a too long degradation cycle, the degradation product produced is accumulated, causing greater adverse effects such as tissue inflammation, edema on the body. However, if the number average molecular weight is less than the value range, the polymer has insufficient mechanical strength, and the fibrous membrane deforms. Meanwhile, with the too small molecular weight, the degradation rate is faster, resulting in too short overall degradation cycle, which is not conducive to the full repair of the tissues.

In a class of this embodiment, in the combination of the poly(lactic-co-glycolic acid) copolymer and polycaprolactone, the mass ratio of the poly(lactic-co-glycolic acid) copolymer to polycaprolactone is between 1:99 and 99:1, such as 1:99, 10:90, 20:80, 30:70, 40:60, 1:1, 60:40, 2:1, 70:30, 1:99, etc., preferably 1:1-2:1.

In the combination of the poly(lactic-co-glycolic acid) copolymer and polycaprolactone, the mass ratio of the poly(lactic-co-glycolic acid) copolymer to polycaprolactone can be any value in the range of between 1:99 and 99:1, where the fibrous membrane material with a mass ratio thereof in the range of between 1:1 and 2:1 can promote the diffusion and growth of the fibroblasts, and the active material can be better dispersed and released.

In a class of this embodiment, the active material comprises gelatin, an epidermal growth factor, a drug, or a combination thereof; for example, a combination of gelatin and epidermal growth factor, or a combination of epidermal growth factor and the drug, or a combination of gelatin and the drug.

The gelatin can improve cell adhesion and growth, and maintain normal cell morphology. The epidermal growth factor can promote the proliferation of epithelial cells and fibroblasts, enhance the viability of epidermal cells, delay the aging of the epidermal cells, and also stimulate the synthesis and secretion of extracellular macromolecules (such as hyaluronic acid and collagen, etc.), and promote tissue repair. The drug can choose traditional anti-inflammatory drugs such as aspirin, benorilate, acetaminophen, levofloxacin, cefradine, and metronidazole, or anti-tumor drugs such as 5-fluorouracil, doxorubicin, cis-platinum, taxol, gemcitabine or capecitabine (these anti-tumor drugs can kill bacteria and viruses that are not conducive to tissue repair in small doses and effectively reduce the probability and degree of inflammatory reactions.

In a class of this embodiment, the drug comprises ciprofloxacin, ciprofloxacin hydrochloride, moxifloxacin, levofloxacin, cefradine, tinidazole, 5-fluorouracil, doxorubicin, cis-platinum, taxol, gemcitabine, capecitabine, or a combination thereof; for example, a combination of ciprofloxacin and ciprofloxacin hydrochloride, a combination of moxifloxacin and levofloxacin, a combination of 5-fluorouracil and doxorubicin, etc. Other arbitrary combinations are not repeated here.

In a class of this embodiment, the drug accounts for 1-50 wt. % of the biodegradable polymer fiber, such as 1%, 2%, 5%, 8%, 10%, 15%, 20%, 25%, 30%, 35%, 40%, 45% or 50%.

The total mass of the drug is within the range of 1-50% of the total mass of the biodegradable polymer fiber. Too much drugs easily form large particle fiber groups. In the process of drug release, the drug is burst and released, thereby increasing local drug concentration, which is not conducive to the growth of tissue cells. At the same time, the large particles are unevenly dispersed, leading to insufficient fiber strength or fiber breakage, which reduces the mechanical strength of the fiber. If the total mass of the drug is less than this range, too little drug is loaded, which is unable to exert normal drug effects. Further, with too little drug, the drug concentration is not maintained during the release process for enough time, which is not conducive to killing harmful substances and affecting the effect of tissue repair.

In a class of this embodiment, the gelatin or the epidermal growth factor accounts for 1-10 wt. % of the biodegradable polymer fiber, for example, 1%, 2%, 5%, 8%, or 10%.

The total mass of the gelatin or epidermal growth factor is within 1-10% because exceeding this range causes excessive active material, which is not conducive to the growth of the tissue cells (excessiveness) while reducing the proportion of the biodegradable polymers and reducing the mechanical strength of the repaired membrane. If the total mass is less than this range, the active material concentration is enough to affect cell proliferation and differentiation, and have no positive effect on the tissue repair.

In another aspect, the disclosure provides a method for preparing the fibrous membrane material for soft tissue repair, and the method comprises:

(1) mixing the biodegradable polymer and the active material in a solvent to obtain a mixed solution;

(2) taking a part of the mixed solution in (1), and introducing the part of the mixed solution to a single-nozzle or multi-nozzle electrostatic spinning apparatus for electrostatic spinning, to obtain the fibrous membrane material for soft tissue repair.

When the biodegradable polymer material comprises a plurality of polymers, the plurality of polymers can be mixed with the active material and the solvent, respectively, and the resulting mixtures are blended and introduced to a single-nozzle or multi-nozzle electrostatic spinning apparatus for electrostatic spinning; or the plurality of polymers is mixed with the active material and the solvent, respectively, and the resulting mixtures are introduced one by one to a multi-nozzle electrostatic spinning apparatus for electrostatic spinning. In the first spinning method, the spinning process is simple, and the fiber diameter is easy to adjust. However, it is necessary to find a good co-solvent when the polymers are mixed by spinning. In addition, the spinning speed is low. In the second spinning method, the multi-nozzle spinning process is complicated, but can spin a plurality of biodegradable polymer fibers at the same time without a co-solvent, and the spinning rate is higher.

When the multi-nozzle electrostatic spinning apparatus is used for electrostatic spinning, a plurality of polymers is loaded with a spot-spaced method, and each polymer is loaded in more syringes. For example, with seven nozzles, the first nozzle, the second nozzle, the sixth nozzle and the seventh nozzle are loaded with a mixed solution of the active material, the biodegradable polymers and the solvent. The third nozzle, the fourth nozzle and the fifth nozzle are loaded with a mixed solution of another biodegradable polymer and the solvent. Alternatively, the first nozzle, the third nozzle, the fifth nozzle and the seventh nozzle are loaded with a mixed solution of the active material, the biodegradable polymer and the solvent, and the second nozzle, the fourth nozzle and the sixth nozzle are loaded with another biodegradable polymer and the solvent. In this way, the two fibers in a system can be mixed more evenly.

In a class of this embodiment, the solvent is N,N-dimethylformamide, acetone, hexafluoroisopropanol or a combination thereof, for example, N,N-dimethylformamide, acetone and a combination thereof, acetone, hexafluoroisopropanol and a combination thereof, and N,N-dimethylformamide, hexafluoroisopropanol and a combination thereof, etc. Other arbitrary combinations are not repeated here.

In a class of this embodiment, the mixing in (1) refers to stirring and mixing at 35-50° C. (for example, 35° C., 40° C., 45° C., or 50° C., etc.).

In a class of this embodiment, the inner diameter of a nozzle for electrostatic spinning in (2) is 0.2-0.8 mm, such as 0.2 mm, 0.4 mm, 0.6 mm or 0.8 mm.

In a class of this embodiment, a voltage for electrostatic spinning is 10-25 kV, such as 10 kV, 12 kV, 13 kV, 14 kV, 15 kV, 16 kV, 18 kV, 20 kV, 22 kV, 24 kV, or 25 kV, preferably 20-25 kV

In a class of this embodiment, a spinning distance for the electrostatic spinning is 5-15 cm, such as 5 cm, 6 cm, 7 cm, 8 cm, 9 cm, 10 cm, 12 cm, 14 cm, or 15 cm, preferably 8-15 cm. In a class of this embodiment, a temperature for the electrostatic spinning in (2) is 20-30° C., such as 20° C., 21° C., 22° C., 23° C., 24° C., 25° C., 26° C., 27° C., 28° C., 29° C. or 30° C., etc.

In a class of this embodiment, an advancing speed of the solution for the electrostatic spinning is 0.2-4 mL/L, for example, 0.2 mL/L, 5 mL/L, 6 mL/L, 7 mL/L, 8 mL/L, 9 mL/L, or 10 mL/L, preferably 0.6-10 mL/L.

In a class of this embodiment, a receiving device for the electrostatic spinning (2) is a metal drum with a diameter of 5-15 cm (for example, 5 cm, 6 cm, 8 cm, 10 cm, 12 cm, 14 cm, or 15 cm, etc.), and the rotating speed is 600-900 rpm (For example, 600 rpm, 650 rpm, 700 rpm,750 rpm, 800 rpm, 850 rpm or 900 rpm, etc.), preferably 800 rpm.

In a class of this embodiment, the fibrous membrane material for soft tissue repair in (2) is further post-processed as follows: the multifunctional fibrous membrane material for soft tissue repair is vacuum-dried at 20-30° C. (for example, 20° C., 21° C., 21° C., 22° C., 23° C., 24° C., 25° C., 26° C., 27° C., 28° C., 29° C. or 30° C. etc.) for 24-72 h (24 h, 30 h, 35 h, 50 h, 60 h or 72 h, etc.).

Optionally, the method for preparing the fibrous membrane material for soft tissue repair comprises the following steps:

(1) mixing the active material, a first biodegradable polymer, and the solvent, to yield a first mixed solution, and then mixing a second biodegradable polymer and the solvent to yield a second mixed solution;

(2) separately taking a part of the first and the second mixed solutions in (1) into 22G syringes, and introducing the part of the first and the second mixed solutions to the multi-nozzle electrostatic spinning apparatus for electrostatic spinning at 20-30° C., where the inner diameter of the nozzle is 0.4 mm; the advancing speed of the solution is 0.6-0.9 mL/h; the spinning voltage is 10-25 kV; the spinning distance is 5-15 cm; the receiving device is a metal drum with a diameter of 5-15 cm, and the rotation speed is 600-900 rpm to obtain the fibrous membrane material for soft tissue repair with a fiber diameter of 0.5-3 μm; and

(3) vacuum-drying the fibrous membrane material for soft tissue repair in (2) at 20-30° C. for 24-72 h.

In another aspect, the disclosure provides a method for preparing a drug delivery system for soft tissue repair, the method comprising applying the fibrous membrane material for soft tissue repair.

Compared with the prior art, the following advantages are associated with the fibrous membrane material for soft tissue repair of the disclosure:

(1) The biodegradable polymer is independently formed into the fiber. The active material is dispersed in the biodegradable polymer fiber. The types and proportions of different biodegradable polymers are adjustable. The proportion of the active material in the biodegradable polymer fiber is adjustable. Electrostatic spinning parameters are adjustable. The diameter and porosity of the biodegradable polymer fiber are adjusted and controlled to further adjust and control the mechanical strength of the fibrous membrane material for soft tissue repair and hence affect the attachment, growth and proliferation of cells (such as fibroblasts).

(2) Different active materials are dispersed into the biodegradable polymer fibers to adjust the type and proportion of the active material added. For example, an appropriate proportion of anti-inflammatory drugs can inhibit soft tissue inflammation. A proper proportion of gelatin (GE) and a proper proportion of the epidermal growth factor can effectively promote the proliferation of special cells (such as the fibroblasts, etc.), accelerate the rate of soft tissue repair, and reduce patient pain. In addition, the active material dispersed in the fibrous membrane material has a better slow and controlled release property.

### BRIEF DESCRIPTION OF THE DIAGRAMS

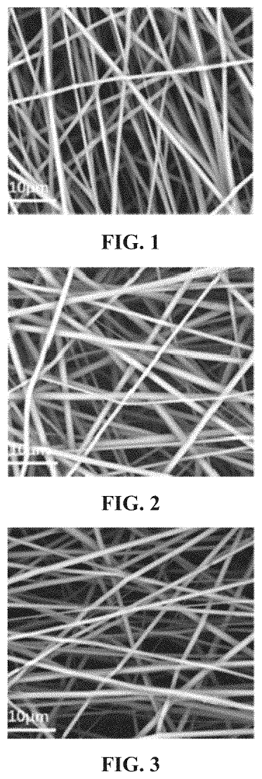

FIG. 1 is a scanning electron microscope (SEM) diagram of a fibrous membrane prepared in Example 1;

FIG. 2 is an SEM diagram of a fibrous membrane with a mass ratio of the poly(lactic-co-glycolic acid) copolymer to polycaprolactone of 2:1 in Example 7;

FIG. 3 is an SEM diagram of a fibrous membrane with a mass ratio of the poly(lactic-co-glycolic acid) copolymer to polycaprolactone of 3:1 in Example 7;

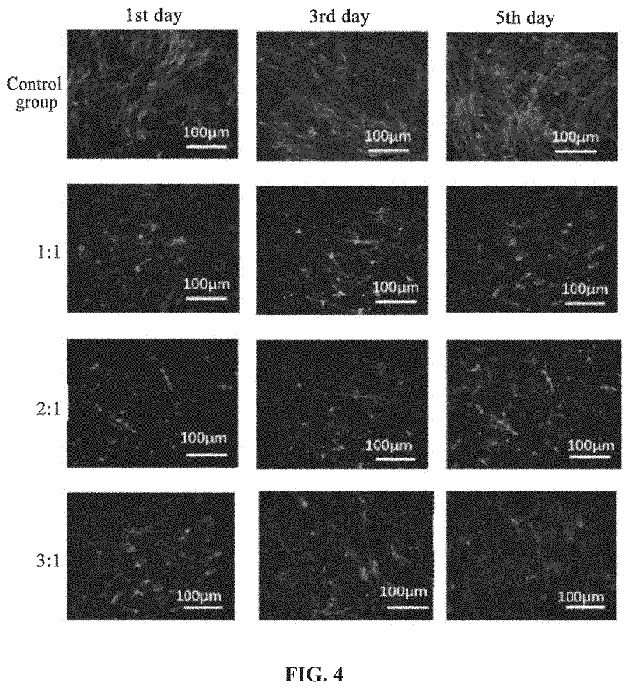

FIG. 4 is cell morphology diagrams of three fibrous membranes prepared in Example 1 and Example 7 for fibroblast culture;

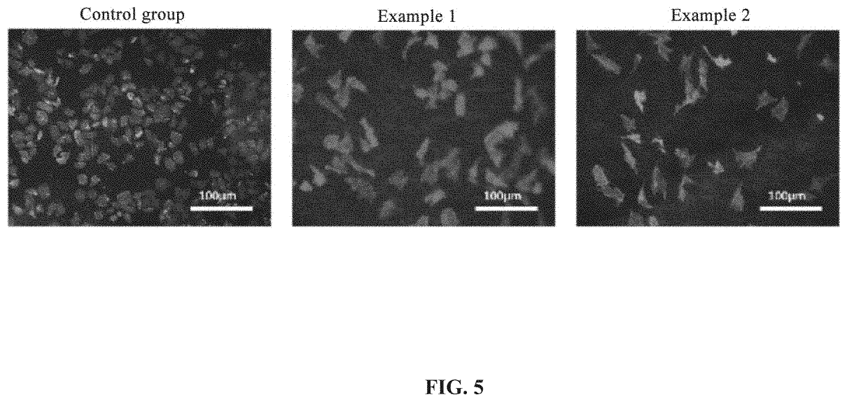

FIG. 5 is cell morphology diagrams of two fibrous membranes prepared in Example 1 and Example 2 for fibroblast culture;

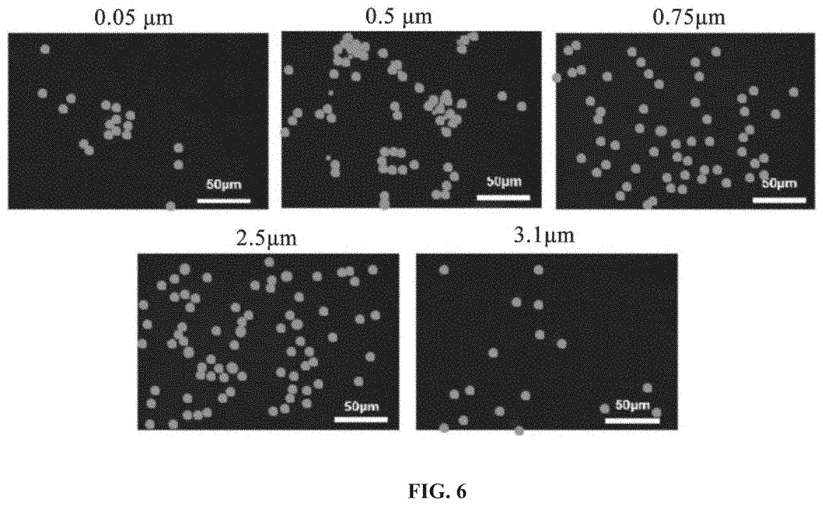

FIG. 6 is cell morphology diagrams of five fibrous membranes prepared in Example 1 and Example 3-6 for fibroblast culture;

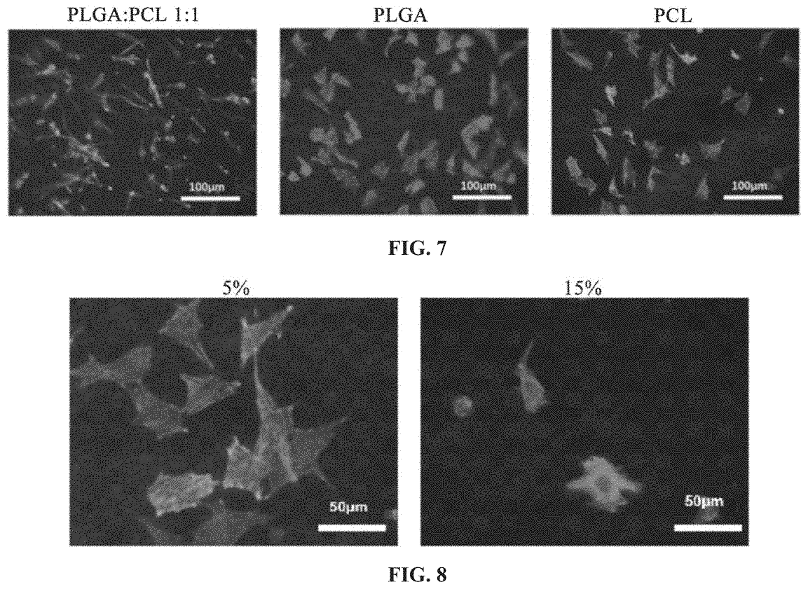

FIG. 7 is cell morphology diagrams of three fibrous membranes prepared in Example 1 and Examples 8-9 for fibroblast culture;

FIG. 8 is cell morphology diagrams of two fibrous membranes prepared in Example 1 and Example 10 for fibroblast culture;

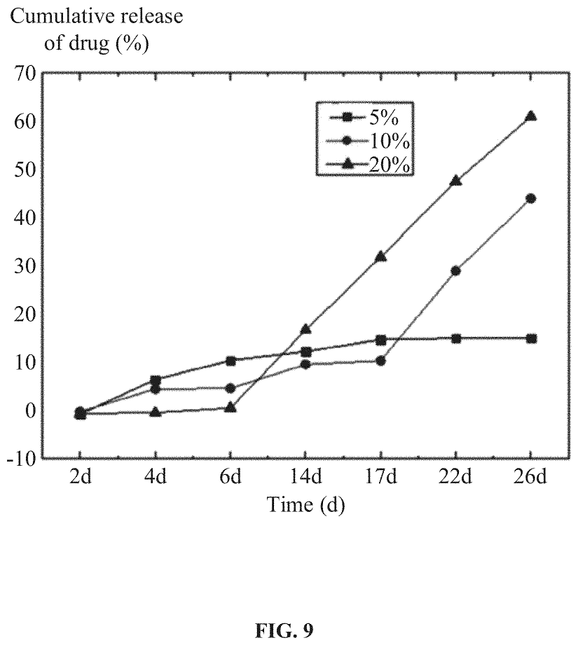

FIG. 9 is a drug release curve of a fibrous membrane material prepared in Example 11;

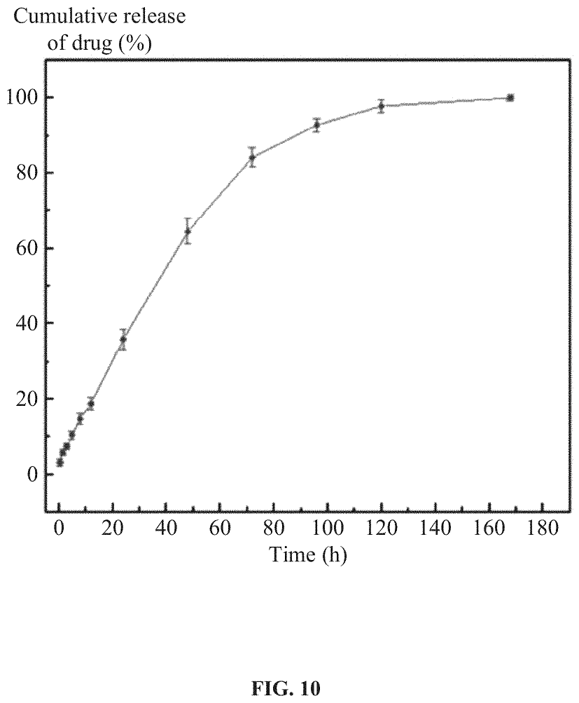

FIG. 10 is a drug release curve of a fibrous membrane material prepared in Example 12; and

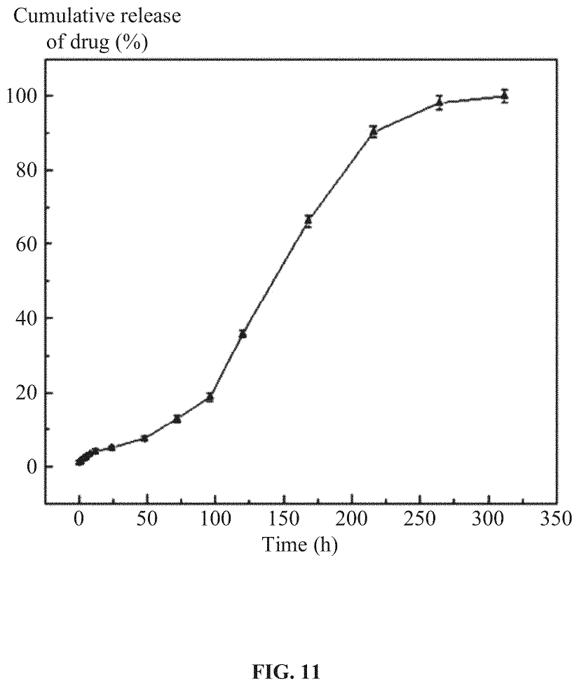

FIG. 11 is a drug release curve of a fibrous membrane material prepared in Example 13.

### DESCRIPTION OF THE INVENTION

To further illustrate, embodiments detailing a fibrous membrane material, a method for preparing the same, and application thereof are described below. It should be noted that the following embodiments are intended to describe and not to limit the disclosure.

Example 1

The disclosure provided a fibrous membrane material for soft tissue repair, comprising a biodegradable polymer (poly(lactic-co-glycolic acid) copolymer (80000 Da) and polycaprolactone (60000 Da) mixed at a mass ratio of 1:1) fiber and an active material of gelatin dispersed therein. The biodegradable polymer fiber had a diameter of 0.75 μm and a porosity of 85%. The mass of the gelatin was 5% of the total mass of the biodegradable polymer fiber. Porosity=(1−p0/p)×100%; where p0 is apparent density of fibrous membrane, p is the density of polymer raw material.

A method for preparing the fibrous membrane material for soft tissue repair was as follows:

(1) stirring and mixing gelatin, poly(lactic-co-glycolic acid) copolymer and N,N-dimethylformamide at 40° C., and then stirring and mixing gelatin, polycaprolactone and N,N-dimethylformamide at 40° C. to obtain two mixed solutions;

(2) mixing and loading the two mixed solutions in (1) into a 22G syringe, introducing the two mixed solutions to a single-nozzle electrostatic spinning apparatus for electrostatic spinning at 25° C., where the inner diameter of a nozzle was 0.4 mm; the advancing speed of the solution was 0.8 mL/h; the spinning voltage was 15 kV; the spinning distance was 10 cm; a receiving device was a metal drum with a diameter of 10 cm, and the rotation speed was 800 rpm to obtain the fibrous membrane material for soft tissue repair; and

(3) vacuum-drying the fibrous membrane material for soft tissue repair in (2) at 25° C. for 48 h.

### Example 2

The disclosure provided a fibrous membrane material for soft tissue repair, comprising a biodegradable polymer (poly(lactic-co-glycolic acid) copolymer (80000 Da) and polycaprolactone (60000 Da) mixed at a mass ratio of 1:1) fiber and an active material of gelatin dispersed therein. The biodegradable polymer fiber had a diameter of 0.75 μm and a porosity of 85%. The mass of the gelatin was 5% of the total mass of the biodegradable polymer fiber.

A method for preparing the fibrous membrane material for soft tissue repair was as follows:

(1) stirring and mixing gelatin, poly(lactic-co-glycolic acid) copolymer and N,N-dimethylformamide at 40° C., and then stirring and mixing gelatin, polycaprolactone and N,N-dimethylformamide at 40° C. to obtain two mixed solutions;

(2) separately loading the two mixed solutions in (1) into 22G syringes, introducing the two mixed solutions to a double-nozzle electrostatic spinning apparatus for electrostatic spinning at 25° C., where the inner diameter of a nozzle was 0.4 mm; the advancing speed of the solution was 0.8 mL/h; the spinning voltage was 18 kV; the spinning distance was 15 cm; a receiving device was a metal drum with a diameter of 10 cm, and the rotation speed was 900 rpm to obtain the fibrous membrane material for soft tissue repair; and

(3) vacuum-drying the fibrous membrane material for soft tissue repair in (2) at 25° C. for 48 h.

### Example 3

The disclosure provided a fibrous membrane material for soft tissue repair, comprising a biodegradable polymer (poly(lactic-co-glycolic acid) copolymer (80000 Da) and polycaprolactone (60000 Da) mixed at a mass ratio of 1:1) fiber and an active material of gelatin dispersed therein. The biodegradable polymer fiber had a diameter of 2.5 μm and a porosity of 65.5%. The mass of the gelatin was 5% of the total mass of the biodegradable polymer fiber.

A method for preparing the fibrous membrane material for soft tissue repair was as follows:

(1) stirring and mixing gelatin, poly(lactic-co-glycolic acid) copolymer and N,N-dimethylformamide at 40° C., and then stirring and mixing gelatin, polycaprolactone and N,N-dimethylformamide at 40° C. to obtain two mixed solutions;

(2) separately loading the two mixed solutions in (1) into 22G syringes, introducing the two mixed solutions to a double-nozzle electrostatic spinning apparatus for electrostatic spinning at 25° C., where the inner diameter of a nozzle was 0.6 mm; the advancing speed of the solution was 0.8 mL/h; the spinning voltage was 13 kV; the spinning distance was 8 cm; a receiving device was a metal drum with a diameter of 10 cm, and the rotation speed was 650 rpm to obtain the fibrous membrane material for soft tissue repair; and

(3) vacuum-drying the fibrous membrane material for soft tissue repair in (2) at 25° C. for 48 h.

### Example 4

The disclosure provided a fibrous membrane material for soft tissue repair, comprising a biodegradable polymer (poly(lactic-co-glycolic acid) copolymer (80000 Da) and polycaprolactone (60000 Da) mixed at a mass ratio of 1:1) fiber and an active material of gelatin dispersed therein. The biodegradable polymer fiber had a diameter of 0.5 μm and a porosity of 89%. The mass of the gelatin was 5% of the total mass of the biodegradable polymer fiber.

A method for preparing the fibrous membrane material for soft tissue repair was as follows:

(1) stirring and mixing gelatin, poly(lactic-co-glycolic acid) copolymer and N,N-dimethylformamide at 40° C., and then stirring and mixing gelatin, polycaprolactone and N,N-dimethylformamide at 40° C. to obtain two mixed solutions;

(2) separately loading the two mixed solutions in (1) into 22G syringes, introducing the two mixed solutions to a double-nozzle electrostatic spinning apparatus for electrostatic spinning at 25° C., where the inner diameter of a nozzle was 0.35 mm; the advancing speed of the solution was 0.8 mL/h; the spinning voltage was 18 kV; the spinning distance was 15 cm; a receiving device was a metal drum with a diameter of 10 cm, and the rotation speed was 850 rpm to obtain the fibrous membrane material for soft tissue repair; and

(3) vacuum-drying the fibrous membrane material for soft tissue repair in (2) at 25° C. for 48 h.

### Example 5

The disclosure provided a fibrous membrane material for soft tissue repair, comprising a biodegradable polymer (poly(lactic-co-glycolic acid) copolymer (80000 Da) and polycaprolactone (60000 Da) mixed at a mass ratio of 1:1) fiber and an active material of gelatin dispersed therein. The biodegradable polymer fiber had a diameter of 3.10 μm and a porosity of 64.3%. The mass of the gelatin was 5% of the total mass of the biodegradable polymer fiber.

Following the method of Example 1, the parameters for the electrostatic spinning were fine-tuned to prepare a polymer fiber having a diameter of 3.10 μm.

### Example 6

The disclosure provided a fibrous membrane material for soft tissue repair, comprising a biodegradable polymer (poly(lactic-co-glycolic acid) copolymer (80000 Da) and polycaprolactone (60000 Da) mixed at a mass ratio of 1:1) fiber and an active material of gelatin dispersed therein. The biodegradable polymer fiber had a diameter of 0.05 μm and a porosity of 93.46%. The mass of the gelatin was 5% of the total mass of the biodegradable polymer fiber.

Following the method of Example 1, the parameters for the electrostatic spinning were fine-tuned to prepare a polymer fiber having a diameter of 0.05 um.

### Example 7

The disclosure provided two fibrous membrane materials for soft tissue repair, comprising a biodegradable polymer (poly(lactic-co-glycolic acid) copolymer (80000 Da) and polycaprolactone (60000 Da) mixed at a mass ratio of 2:1 and 3:1, respectively) fiber and an active material of gelatin dispersed therein. The biodegradable polymer fiber had a diameter of 0.75 μm and a porosity of 84.55%. The mass of the gelatin was 5% of the total mass of the biodegradable polymer fiber.

Following the method of Example 1, the parameters for the electrostatic spinning were fine-tuned to prepare a polymer fiber having a diameter of 0.75 um.

### Example 8

The disclosure provided a fibrous membrane material for soft tissue repair, comprising a biodegradable polymer (poly(lactic-co-glycolic acid) copolymer (80000 Da)) fiber and an active material of gelatin dispersed therein. The biodegradable polymer fiber had a diameter of 0.75 μm and a porosity of 85.12%. The mass of the gelatin was 5% of the total mass of the biodegradable polymer fiber.

Following the method of Example 1, the parameters for the electrostatic spinning were fine-tuned to prepare a polymer fiber having a diameter of 0.75 μm.

### Example 9

The disclosure provided a fibrous membrane material for soft tissue repair, comprising a biodegradable polymer (polycaprolactone (60000 Da)) fiber and an active material of gelatin dispersed therein. The biodegradable polymer fiber had a diameter of 0.75 μm and a porosity of 85.33%. The mass of the gelatin was 5% of the total mass of the biodegradable polymer fiber.

Following the method of Example 1, the parameters for the electrostatic spinning were fine-tuned to prepare a polymer fiber having a diameter of 0.75

### Example 10

The disclosure provided a fibrous membrane material for soft tissue repair, comprising a biodegradable polymer (poly(lactic-co-glycolic acid) copolymer (80000 Da) and polycaprolactone (60000 Da) mixed at a mass ratio of 1:1) fiber and an active material of gelatin dispersed therein. The biodegradable polymer fiber had a diameter of 0.75 μm and a porosity of 84.15%. The mass of the gelatin was 15% of the total mass of the biodegradable polymer fiber.

Following the method of Example 1, the parameters for the electrostatic spinning were fine-tuned to prepare a polymer fiber having a diameter of 0.75

### Example 11

The disclosure provided three fibrous membrane materials for soft tissue repair, comprising a biodegradable polymer (poly(lactic-co-glycolic acid) copolymer (60000 Da)) fiber and an active material of paclitaxel dispersed therein. The biodegradable polymer fiber had a diameter of 0.75 μm and a porosity of 85%. In the three fibrous membrane materials, the mass of the paclitaxel was 5%, 10%, and 20% of the total mass of the biodegradable polymer fiber, respectively.

Following the method of Example 1, the parameters for the electrostatic spinning were fine-tuned to prepare a polymer fiber having a diameter of 0.75 μm.

### Example 12

The disclosure provided a fibrous membrane material for soft tissue repair, comprising a biodegradable polymer (poly(lactic-co-glycolic acid) copolymer (60000 Da)) fiber and an active material of 5-fluorouracil dispersed therein. The biodegradable polymer fiber had a diameter of 0.75 μm and a porosity of 85%. The mass of the 5-fluorouracil was 10% of the total mass of the biodegradable polymer fiber.

Following the method of Example 1, the parameters for the electrostatic spinning were fine-tuned to prepare a polymer fiber having a diameter of 0.75 μm.

### Example 13

The disclosure provided a fibrous membrane material for soft tissue repair, comprising a biodegradable polymer (poly(lactic-co-glycolic acid) copolymer (60000 Da)) fiber and an active material of cefradine dispersed therein. The biodegradable polymer fiber had a diameter of 0.75 μm and a porosity of 84.56%. The mass of the cefradine was 10% of the total mass of the biodegradable polymer fiber.

Following the method of Example 1, the parameters for the electrostatic spinning were fine-tuned to prepare a polymer fiber having a diameter of 0.75 μm.

Evaluating the test:

(1) SEM test:

The three fibrous membrane material for soft tissue repairs in Example 1 and Example 7 were scanned with an electron microscope, and results were shown in FIGS. 1-3 (FIG. 1 was the fibrous membrane prepared in Example 1; FIG. 2 was the fibrous membrane with a mass ratio of the poly(lactic-co-glycolic acid) copolymer to polycaprolactone of 2:1 in Example 7, and FIG. 3 was the fibrous membrane with a mass ratio of the poly(lactic-co-glycolic acid) copolymer to polycaprolactone of 3:1 in Example 7). It can be seen from FIGS. 1-3 that the fiber diameters with the three different mass rations of the poly(lactic-co-glycolic acid) copolymer to polycaprolactone were relatively uniform, and were basically maintained at about 0.75 um.

(2) Cell Culture Test:

1. Performing fibroblast culture on the three fibrous membranes in Example 1 and Example 7, where the operation method was as follows: isolating Hs 865.Sk (ATCC-CRL-7601) cells on a culture plate with a protease enzymolysis method, centrifuging at 1000 rpm for 5 min, and adding a 10% (v/v) fetal bovine serum and a 1% (v/v) chloromycetin/streptomycin to a DMEM/F12 1:1 medium. Cells were suspended, planted and fixed in the membrane. The cells were cultured in the DMEM/F12 1:1 and the 10% fetal bovine serum (Hyclone) at 37° C. and 5% CO₂ for 5 days, to produce proliferation and adhesion. The distribution of fibroblasts cultured on the fibrous membrane on the 1, 3, and 5 days after culture was shown in FIG. 4 (the cells were fluorescence-stained with a cell fluorescence staining method). In the soft tissue repair membranes with different ratios of the poly(lactic-co-glycolic acid) copolymer to polycaprolactone, the cells grew well with good morphology. The number of cells was gradually increased from the first day. In the repaired membranes with the mass ratio of the poly(lactic-co-glycolic acid) copolymer to polycaprolactone being 1:1 and 3:1, the cells grew well. However, the cells obviously contiguously grew, that is, the cell grew too fast, leading to tissue adhesion. In the repaired membrane with the ratio of 2:1, the cells had a tendency to grow fast, but not too fast. The cells grew stably without obvious contiguous growth and excessive proliferation. Therefore, the appropriate ratio of the poly(lactic-co-glycolic acid) copolymer to polycaprolactone is conducive to the normal growth of tissue cells.

2. Performing the fibroblast culture on the two fibrous membranes in Example 1 and Example 2, where the operation method was as above. On the fifth day of culture, the distribution of the fibroblasts on the fibrous membrane was shown in FIG. 5 (the cells were fluorescence-stained with the cell fluorescence staining method). It could be seen from FIG. 5 that single-nozzle spinning cell culture results were better than double-nozzle spinning cell culture results. The morphology, size and number of the cells were all closer to the real growth of the cells.

3. Performing the fibroblast culture on the two fibrous membranes in Example 1 and Examples 3-6, where the operation method was as above. On the fifth day of culture, the distribution of the fibroblasts on the fibrous membrane was shown in FIG. 6 (the cells were fluorescence-stained with the cell fluorescence staining method). It could be seen from FIG. 6 that when the diameter of the repaired membrane fibers was in the proper range (0.1-3 μm), the cells grew well. With an increase in the diameter, the number of the cells increased significantly, and the cell morphology grew well. Too fine fibers easily led to the aggregation of the cells, which is not conducive to the proliferation of the cells, resulting in too small cell number. However, too thick fibers result in a decline in the attachment of the cells, which is not conducive to the good morphology of the cells.

4. Performing the fibroblast culture on the two fibrous membranes in Example 1 and Examples 8-9, where the operation method was as above. On the fifth day of culture, the distribution of the fibroblasts on the fibrous membrane was shown in FIG. 7 (the cells were fluorescence-stained with the cell fluorescence staining method). It could be seen from FIG. 7 that in poly(lactic-co-glycolic acid) copolymer, the cell grew best with good cell morphology. The cells were relatively dispersed and uniform, and mutually involved, which is conducive to the formation of new tissues. In pure poly(lactic-co-glycolic acid) copolymer, the cells were larger but scattered and independent without connection. In pure polycaprolactone, the number of cells was significantly reduced, and the cells are more scattered and independent, resulting in the poor effect of the tissue repair.

5. Performing the fibroblast culture on the two fibrous membranes in Example 1 and Example 10, where the operation method was as above. On the fifth day of culture, the distribution of the fibroblasts on the fibrous membrane was shown in FIG. 8 (the cells were fluorescence-stained with the cell fluorescence staining method). It could be seen from FIG. 8 that in the repaired membranes with the mass ratio of the poly(lactic-co-glycolic acid) copolymer to polycaprolactone of 1:1, the cells cultured with the fibrous membrane with 5% of gelatin content had complete morphology and the larger number of cells. However, when the gelatin content was 15%, the number of cells decreased, and the cell morphology was obviously not as good as that of 5% cells due to the excessive gelatin content. The effect of the active material was basically not enhanced, but the attachment of the cells to the fibrous membrane was reduced, leading to adverse effect on the proliferation and differentiation of the cells.

(3) Drug release test:

The fibrous membranes in Examples 11-13 were tested with drug release to draw a release curve. The method was as follows:

(1) putting each fibrous membrane into a centrifuge tube containing 10 mL of a fresh PBS solution;

(2) putting the centrifuge tube into an air bath constant-temperature shaker at 37° C. with the speed of the shaker of 100 rpm, taking out 1 mL of the release solution and replenishing the same amount of the fresh PBS solution at a specified time interval;

(3) measuring 1 mL of the release solution with an ultraviolet-visible spectrophotometer, and determining the amount of the released drug according to a standard curve, where the results were measured in parallel for 5 times, and the measured drug release was expressed as an average value±standard deviation.

The results were shown in FIGS. 9-11 (FIG. 9 was the drug release curve of Example 11, FIG. 10 was the drug release curve of Example 12, and FIG. 11 was the drug release curve of Example 13).

FIG. 8 showed the release curve of taxol with different mass ratios. In the early stage of release, taxol maintained a low release rate. After a period of sustained release, the release rate accelerated and rose steadily. At the same time, with the increase of taxol, the gentle release cycle of taxol gradually decreased.

It could be seen from FIG. 9 that the release of 5-fluorouracil rose steadily at a constant rate in the early stage, and tended to be linear. After 90 h, the release rate of 5-fluorouracil began to gradually slow down until the drug release was complete.

It could be seen from FIG. 10 that the release cycle of cefradine was about 360 h. The release of cefradine tended to be flat in the early and late stages. At about 75 h, the release rate gradually increased, then began to be stable and fast, and gradually slowed down at about 230 h, and began to release slowly until the drug was completely released.

It will be obvious to those skilled in the art that changes and modifications may be made, and therefore, the aim in the appended claims is to cover all such changes and modifications.

What is claimed is:

1. A fibrous membrane material for soft tissue repair, comprising a biodegradable polymer fiber and an active material dispersed in the biodegradable polymer fiber.

2. The fibrous membrane material of claim 1, wherein the biodegradable polymer fiber has a diameter of 0.1-3 μm; and the biodegradable polymer fiber has a porosity of 65-90%.

3. The fibrous membrane material of claim 1, wherein the biodegradable polymer fiber comprises a biodegradable polymer selected from the group consisting of polylactic acid (PLA), poly(lactic-co-glycolic acid) copolymer, polyethylene glycol (PEG), poly(p-dioxanone), polycaprolactone, poly(L-lactide-co-caprolactone), a triblock copolymer PLA-b-PEG-b-PLA, and a combination thereof.

4. The fibrous membrane material of claim 2, wherein the biodegradable polymer fiber comprises a biodegradable polymer selected from the group consisting of polylactic acid (PLA), poly(lactic-co-glycolic acid) copolymer, polyethylene glycol (PEG), poly(p-dioxanone), polycaprolactone, poly(L-lactide-co-caprolactone), a triblock copolymer PLA-b-PEG-b-PLA, and a combination thereof.

5. The fibrous membrane material of claim 3, wherein:
a number average molecular weight of the polylactic acid is 8000-70000 Da;
a number average molecular weight of the poly(lactic-co-glycolic acid) copolymer is 40000-100000 Da;
a number average molecular weight of the polyethylene glycol is 1000-20000 Da;
a number average molecular weight of the poly(p-dioxanone) is 60000-250000 Da;
a number average molecular weight of the polycaprolactone is 6000-100,000 Da;
a molar ratio of lactide units to caprolactone units of the poly(L-lactide-co-caprolactone) is between 1:99 and 50:50, and an average molecular weight of the poly(L-lactide-co-caprolactone) is 35000-85000 Da; and
an average molecular weight of the triblock copolymer PLA-b-PEG-b-PLA is 60000-100000 Da.

6. The fibrous membrane material of claim 4, wherein:
a number average molecular weight of the polylactic acid is 8000-70000 Da;
a number average molecular weight of the poly(lactic-co-glycolic acid) copolymer is 40000-100000 Da;
a number average molecular weight of the polyethylene glycol is 1000-20000 Da;
a number average molecular weight of the poly(p-dioxanone) is 60000-250000 Da;
a number average molecular weight of the polycaprolactone is 6000-100,000 Da;
a molar ratio of lactide units to caprolactone units of the poly(L-lactide-co-caprolactone) is between 1:99 and 50:50, and an average molecular weight of the poly(L-lactide-co-caprolactone) is 35000-85000 Da; and
an average molecular weight of the triblock copolymer PLA-b-PEG-b-PLA is 60000-100000 Da.

7. The fibrous membrane material of claim 3, wherein in a combination of the poly(lactic-co-glycolic acid) copolymer and the polycaprolactone, a mass ratio of the poly(lactic-co-glycolic acid) copolymer to the polycaprolactone is between 1:99 and 99:1.

8. The fibrous membrane material of claim 4, wherein in a combination of the poly(lactic-co-glycolic acid) copolymer and the polycaprolactone, a mass ratio of the poly(lactic-co-glycolic acid) copolymer to the polycaprolactone is between 1:99 and 99:1.

9. The fibrous membrane material of claim 7, wherein in a combination of the poly(lactic-co-glycolic acid) copolymer and the polycaprolactone, a mass ratio of the poly(lactic-co-glycolic acid) copolymer to the polycaprolactone is between 1:1 and 2:1.

10. The fibrous membrane material of claim 8, wherein in a combination of the poly(lactic-co-glycolic acid) copolymer and the polycaprolactone, a mass ratio of the poly(lactic-co-glycolic acid) copolymer to the polycaprolactone is between 1:1 and 2:1.

11. The fibrous membrane material of claim 1, wherein the active material comprises gelatin, an epidermal growth factor, a drug, or a combination thereof; the drug comprises ciprofloxacin, ciprofloxacin hydrochloride, moxifloxacin, levofloxacin, cefradine, tinidazole, 5-fluorouracil, doxorubicin, cis-platinum, taxol, gemcitabine, capecitabine, or a combination thereof; the drug accounts for 1-50 wt. % of the biodegradable polymer fiber; and the gelatin or the epidermal growth factor accounts for 1-10 wt. % of the biodegradable polymer fiber.

12. A method for preparing the fibrous membrane material for soft tissue repair of claim 1, the method comprising:
(1) mixing a biodegradable polymer and the active material in a solvent to obtain a mixed solution; and
(2) taking a part of the mixed solution in (1), and introducing the part of the mixed solution to a single-nozzle or multi-nozzle electrostatic spinning apparatus for electrostatic spinning, to obtain the fibrous membrane material for soft tissue repair.

13. The method of claim 12, wherein the solvent is N,N-dimethylformamide, acetone, hexafluoroisopropanol, or a combination thereof;
in (1), the biodegradable polymer and the active material are mixed in the solvent at 35-50° C. under stirring;
in (2), an inner diameter of a nozzle of the single-nozzle or multi-nozzle electrostatic spinning apparatus is 0.2-0.8 mm;
a voltage during electrostatic spinning is 10-25 kV;
a spinning distance during the electrostatic spinning is 5-15 cm;
a temperature for the electrostatic spinning is 20-30° C.;
an advancing speed of the mixed solution during the electrostatic spinning is 0.2-4 mL/L; and
a receiving device during the electrostatic spinning is a metal drum with a diameter of 5-15 cm, and a rotation speed of the metal drum is 600-900 rpm.

14. The method of claim 12, wherein after 2), the fibrous membrane material for soft tissue repair is vacuum-dried at 20-30° C. for 24-72 h.

15. The method of claim 13, wherein after 2), the fibrous membrane material for soft tissue repair is vacuum-dried at 20-30° C. for 24-72 h.

16. A method for preparing a drug delivery system for soft tissue repair, the method comprising applying the fibrous membrane material for soft tissue repair of claim 1.
